(19) United States
(12) Patent Application Publication
(10) Pub. No.: US 2012/0149766 A1
(43) Pub. Date: Jun. 14, 2012

(54) DIETARY AND PHARMACEUTICAL COMPOSITIONS COMPRISING A SAGE EXTRACT CONTAINING A MIXTURE OF TRICYCLIC DITERPENES AND THEIR DERIVATIVES AND THEIR USES

(75) Inventors: Ann FOWLER, Rheinfelden (CH); Claus KILPERT, Mannheim (DE); Goede SCHUELER, Eimeldingen (DE); Christof WEHRLI, Witterswil (CH); Adrian WYSS, Basel (CH)

(73) Assignee: DSM IP ASSETS B.V., Heerlen (NL)

(21) Appl. No.: 13/366,575

(22) Filed: Feb. 6, 2012

Related U.S. Application Data
(63) Continuation of application No. 12/515,328, filed on May 18, 2009.

(30) Foreign Application Priority Data
Nov. 24, 2006 (EP) 06024383.9

(51) Int. Cl. A61K 31/366; A61P 25/20; A61P 25/22; A61P 25/00; A61P 25/24

(52) U.S. Cl. 514/453

(57) ABSTRACT

The present invention refers to a sage extract containing a mixture of tricyclic diterpene (derivative)s of the formulae I to III, preferably to a sage extract containing a mixture of tricyclic diterpene (derivative)s of the formulae I to IV, more preferably to a sage extract containing a mixture of tricyclic diterpene (derivative)s of the formulae I to V, for use as medicaments for the treatment of a disorder connected to reduced neurotransmission, as well as to dietary and pharmaceutical compositions and their uses.

This application is a continuation of U.S. application Ser. No. 12/515,328 filed May 18, 2009, which is the U.S. national phase of International Application No. PCT/EP2007/010134, filed 22 Nov. 2007, which designated the U.S. and claims priority to European Application No. 06024383.9, filed 24 Nov. 2006, the entire contents of each of which are hereby incorporated by reference.

### BRIEF DESCRIPTION OF THE INVENTION

The present invention refers to a sage extract comprising a mixture of tricyclic diterpenes and their derivatives for use as medicaments, especially for the treatment of disorders connected to impaired, i.e. reduced, neurotransmission, as well as to dietary and pharmaceutical compositions containing such a sage extract and their uses.

### BACKGROUND OF THE INVENTION

It is well known that impaired neurotransmission, e.g. low neurotransmitter levels, is connected to mental diseases, such as depression and generalized anxiety disorder (GAD), and increased susceptibility to stress.

Compounds that increase neurotransmitter levels in the brain and thus enhance their transmission, can exhibit antidepressant properties as well as beneficial effects on a variety of other mental disorders (Neurotransmitters, drugs and brain function, R. A. Webster (ed), John Wiley & Sons, New York, 2001, p. 187-211, 289-452, 477-498). The main neurotransmitters are serotonin, dopamine, noradrenaline (=norepinephrine), acetylcholine, glutamate, gamma-amino-butyric acid, as well as neuropeptides. Those neurotransmitters of particular relevance to mood-related disorders include serotonin, noradrenaline and dopamine. Enhanced or prolonged neurotransmission is achieved by increasing the concentration of the neurotransmitter in the synaptic cleft, through inhibition of re-uptake into the pre-synaptic nerve ending, or by preventing neurotransmitter catabolism by inhibition of degrading enzymes such as monoamine oxidases (MAOs) -A and -B.

### Antidepressants and Mood-Related Disorders

Tricyclic antidepressant compounds (TCAs), such as imipramine, amitriptyline and clomipramine, inhibit the re-uptake of serotonin and noradrenaline. They are widely regarded as among the most effective antidepressants available, but they have a number of disadvantages because they additionally interact with muscarinic acetylcholine-, histamine- and serotonin-receptors. Side effects resulting from such activities include dry mouth, blurred vision, constipation and urinary retention, in addition to postural hypotension. Most importantly, TCAs are not safe when taken in overdose, frequently showing acute cardiotoxicity.

Another class of antidepressant drugs are the so-called SSRIs (selective serotonin re-uptake inhibitors) including fluoxetine, paroxetine, sertraline, citalopram and fluvoxamine, that block the serotonin transporter (SERI), a high affinity sodium chloride-dependent neurotransmitter transporter that terminates serotonergic neurotransmission by re-uptake of serotonin. They have been proven as effective in the treatment of depression and anxiety as TCAs, but are usually better tolerated. These medications are typically started at low dosages and are increased until they reach a therapeutic level. A common side effect is nausea. Other possible side effects include decreased appetite, dry mouth, sweating, infection, constipation, tremor, yawning, sleepiness and sexual dysfunction.

In addition, compounds that prevent the catabolism of neurotransmitters more broadly by inhibiting MAOs -A and -B exhibit antidepressant effects. MAOs catalyse the oxidation of amine group-containing neurotransmitters, such as serotonin, noradrenaline and dopamine.

Furthermore, modulators of neurotransmission exert pleiotropic effects on mental and cognitive functions.

There is a need for compounds for the treatment or prevention of mental diseases and/or disorders which do not show the negative side-effects of known antidepressants. Many patients are interested in alternative therapies which could minimise the side effects associated with high doses of drugs and yield additive clinical benefits. Severe depression is a long-lasting and recurring disease, which is usually poorly diagnosed. Furthermore, many patients suffer from mild or moderately severe depression. Thus, there is an increasing interest in the development of compounds, as well as pharmaceutical and/or dietary compositions, that may be used to treat mental diseases/disorders or to prevent the development of mental diseases/disorders, such as depression and dysthymia, in people at risk, to stabilise mood and achieve emotional balance.

Patients often suffer either as comorbidity to depression, or alone, from GAD, which is a highly prevalent anxiety condition and chronic illness in primary care (˜10% of patients) (Wittchen et al 2005. Eur. Neuropsycho, 15:357-376). Patients present themselves to their primary care physician with multiple physical symptoms. GAD is characterised by chronic tension, and anxious worrying and tension (>6 months), which are disabling and uncontrollable, and accompanied by a characteristic hypervigilance syndrome (including restlessness, muscle tension and sleep problems). If untreated, GAD runs a chronic, fluctuating course and tends to become increasingly severe with age. GAD patients suffer from subsyndromal depression and contribute to the highest overall direct and indirect health economic burden of all anxiety and depressive disorders. Despite high GAD incidence, few sufferers are diagnosed, prescribed medication, or receive psychiatric referral; simple diagnostic tools to aid patient recognition and monitoring are needed. Regardless of specific diagnosis, physicians require effective GAD-symptom treatments. SSRIs such as paroxetine are effective for GAD treatment (Stocchi et al., 2003 J Clin Psych, 63(3): 250-258). Also, systematic reviews and placebo-controlled RCTs (Randomised Clinical Trials) indicate that some SSRIs (escitalopram, paroxetine and sertraline), the SNRI (Selective Noradrenaline Reuptake Inhibitor) venlafaxine, some benzodiazepines (alprazolam and diazepam), the TCA imipramine, and the 5-HT_{1A} partial agonist, buspirone, are all efficacious in acute treatment. In general, the effect of treatment is often moderate and symptoms reappear when the treatment period is discontinued. Therefore, a continuous long-term treatment or prevention with compounds which have fewer side effects than SSRIs and can be taken over long time periods might be favourable over drug treatment.

Mood disorders and occupational stress can lead to sleep disorders, insomnia, low sleep quality and general disturbances in circadian rhythms (so-called biorhythms); such conditions are often chronic and persistent in nature. Also, dysregulation of circadian rhythms induced by long-haul flights (jet-lag) and shift-work can cause similar symptoms and distress. Therefore, treatment with dietary supplementation to maintain a normal circadian rhythm (that a human or animal is used to) and/or to alleviate and prevent symptoms associated with a disturbed circadian rhythm, such as impairment of cognitive function and memory and mental and physical fatigue, thus improving the overall quality of life and benefiting the vital energy of a person in need thereof, would be most desirable.

### BRIEF DESCRIPTION OF THE DRAWINGS

FIG. 1 illustrates the compounds I to V of the sage extracts used in the invention.

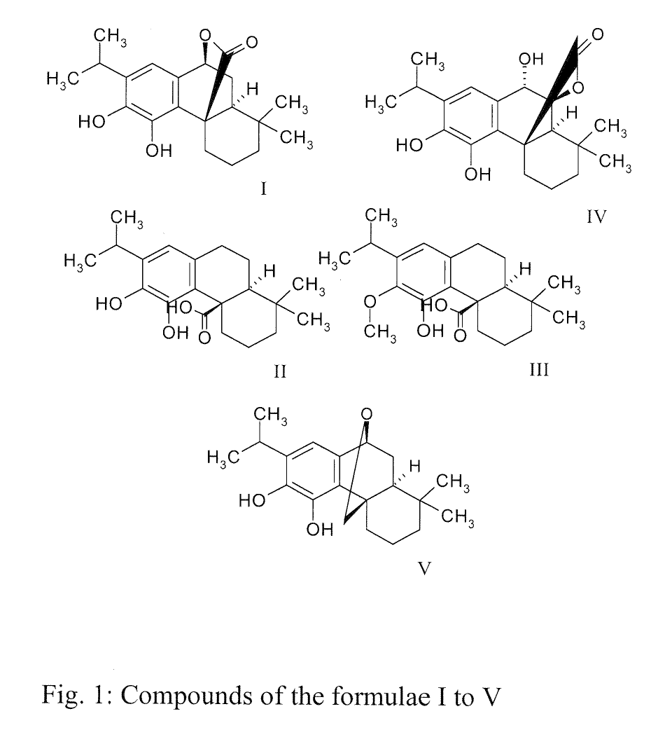

### DETAILED DESCRIPTION OF THE INVENTION

### Sage Extracts and Their Preferred Components

It has been found, in accordance with this invention, that a sage extract comprising the following tricyclic diterpene (derivative)s of the formulae I to III, i.e. carnosol (compound of formula I), carnosic acid (compound of formula II) and carnosic acid 12-methyl ether (compound of formula III) can be used in medicaments for the treatment of a disorder connected to reduced neurotransmission.

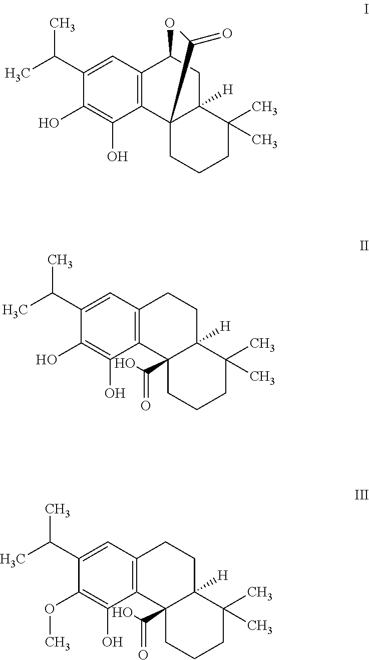

Optionally rosmanol (compound of the formula IV) and/or 20-deoxocarnosol (compound of the formula V) may also be present.

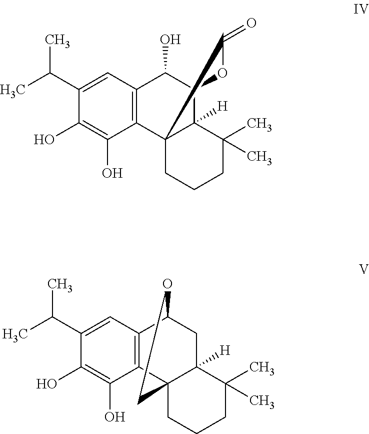

Especially preferred are those sage extracts that (beside carnosol, carnosic acid and carnosic acid 12-methyl ether) additionally contain rosmanol.

The amounts of these tricyclic diterpenes in the sage extract may vary in the range of from 0.1 to 15 weight-% (preferably from 1 to 12 weight-%, more preferably from 9 to 11 weight-%) for carnosic acid, in the range of from 0.01 to 10 weight-% (preferably from 2 to 8 weight-%, more preferably from 2 to 5 weight-%) for carnosol and in the range of from 0.1 to 7 weight-% (preferably from 0.2 to 6 weight-%, more preferably from 3 to 5 weight-%) for carnosic acid 12 methyl ether, based on the total weight of the sage extract, whereby carnosic acid is preferably the main component.

If rosmanol is present its amount may vary in the range of from 0.1 to 1.0 weight-%, preferably from 0.1 to 0.7 weight-%, more preferably from 0,1 to 0.5 weight-%.

If 20-deoxo-carnosol is present its amount may be below 1.0 weight-%, preferably it may vary in the range of from 0.01 to 1.0 weight-%, more preferably from 0.05 to 0.8 weight-%, most preferably from 0.07 to 0.5 weight-%.

The term “sage extract” means any extract of sage containing the tricyclic diterpene (derivative)s I, II and III, preferably containing the tricyclic diterpene (derivative)s I, II, III and IV, more preferably containing the tricyclic diterpene (derivative)s I, II, III, IV and V. Suitable sage species are Salvia officinalis, Salvia miltiorrhiza, Salvia lanigera, Salvia canariensis, Salvia fruticosa, Salvia mellifera, Salvia tomentosa, Salvia deserta, Salvia przewalskii, and Salvia sclarea. Preferred is Salvia officinalis.

Sage extracts suitable for the uses of the present invention are commercially available from, for example, FLAVEX Naturextrakte GmbH, Rehlingen, Germany. The following two extracts were used in the Examples (see later):

(1) “Salbei Extrakt, Typ Nr, 063.001”, containing 0.06 weight-% of carnosol, 0.14 weight-% of carnosic acid and 0.36 weight-% of carnosic acid 12-methyl ether, based on the total weight of the sage extract (measured by HPLC-UV at 210 nm with the pure substances as reference).

(2) “Salbei Antioxidans Extrakt, Typ Nr. 063.007”, containing 3.50 weight-% of carnosol, 10.70 weight-% of carnosic acid, 4.30 weight-% of carnosic acid 12-methyl ether, 0.3 weight-% of 20-deoxocarnosol and 0.3 weight-% of rosmanol, based on the total weight of the sage extract (measured by HPLC-UV at 210 nm with the pure substances as reference).

Another sage extract suitable for the purposes of the present invention contains 7.44 weight-% of carnosol, 1.56 weight-% of carnosic acid, 0.23 weight-% of carnosic acid 12-methyl ether, below 0.1 weight-% of 20-deoxocarnosol and 0.41 weight-% of rosmanol, based on the total weight of the sage extract (measured by HPLC-UV at 210 nm with the pure substances as reference).

Sage extracts suitable for the uses of the present invention may be manufactured according to one of the following two procedures:

Procedure 1

Dried Salvia officinalis leaves (raw material) are mechanically reduced to small pieces, which are then placed in a pressure-stable extraction vessel. Super-critical CO₂ is then passed through the extraction vessel at a temperature in the range of from 25 to 75° C. (preferably at a temperature of ca. 50° C.) and at a high pressure (preferably 280 bar) in the presence of a small amount of ethanol, thus enabling the lipophilic compounds to dissolve. In the separator the dissolved compounds (the extract) are separated by a pressure reduction to a pressure in the range of from 40 to 80 bar (preferably by a pressure reduction to 60 bar), at a temperature in the range of from 15 to 45° C. (preferably at a temperature of 30° C.). The CO₂ may be recycled back into the process. Approximately 1 kg of a sage extract containing:
carnosol (preferably in an amount of from 2 to 5 weight-%, based on the total weight of the sage extract),
carnosic acid (preferably in an amount of from 7 to 15 weight-%, based on the total weight of the sage extract),
carnosic acid 12-methyl ether (preferably in an amount of from 2 to 7 weight-%, based on the total weight of the sage extract),
rosmanol (preferably in an amount of from 0.1 to 1 weight-%, based on the total weight of the sage extract) and
20-deoxocarnosol (preferably in an amount of from 0.1 to 1 weight-%, based on the total weight of the sage extract)
   may be obtained from 14 to 20 kg of dried Salvia officinalis leaves.

Procedure 2

700 g of dried Salvia officinalis leaves are extracted by methanol and then evaporated to dryness. Subsequent solvent-solvent partitionings by use of hexane:H₂O followed by methyl tert-butyl ether (MTBE):H₂O are performed. The MTBE partition is evaporated to dryness. 40 g of this extract may be obtained, which yields enriched amounts of abietanes and triterpenes (carnosol, preferably in an amount of from 5 to 10 weight-%; carnosic acid, preferably in an amount of from 1 to 3 weight-%; carnosic acid 12-methyl ether, preferably in an amount of from 0.05 to 0.5 weight-%; rosmanol, preferably in an amount of from 0.1 to 1 weight-%; and 20-deoxocarnosol, preferably in an amount below 0.1 weight-%; all amounts based on the total weight of the sage extract). If desired, MPLC chromatography can subsequently be applied to remove oleanolic acid, betulinic acid, or ursolic acid from the extract (yield 25 g).

The person skilled in the art knows other extraction methods which yield a sage extract enriched with carnosic acid, carnosol and carnosic acid 12-methyl ether and preferably also in rosmanol and/or 20-deoxocarnosol.

“Carnosol” means the racemic mixture as well as pure (4aR,9S,10aS)-carnosol or pure (4aS,9R,10aR)-carnosol or any mixture or diastereoisomer of them. (4aR,9S,10aS)-carnosol is preferred.

“Carnosic acid” means the racemic mixture as well as pure (4aR,10aS)-carnosic acid or pure (4aS,10aR)-carnosic acid or any mixture or diastereoisomer of them. Preferred is (4aR,10aS)-carnosic acid.

“Carnosic acid 12-methyl ether” means the racemic mixture as well as pure (4aR,10aS)-carnosic acid 12-methyl ether or pure (4aS,10aR)-carnosic acid 12-methyl ether or any mixture or diastereoisomer of them. Preferred is (4aR,10aS)-carnosic acid 12-methyl ether.

“Rosmanol” means the racemic mixture as well as pure (4aR,9S,10aS)-rosmanol or pure (4aS,9R,10aR)-rosmanol or any mixture or diastereoisomer of them. (4aR,9S,10aS)-rosmanol is preferred.

“20-Deoxo-carnosol” means the racemic mixture as well as pure (4aR,9S,10aS)-20-deoxocarnosol or pure (4aS,9R,10aR)-20-deoxo-carnosol or any mixture or diastereoisomer of them. (4aR,9S,10aS)-20-deoxo-carnosol is preferred.

### Uses of Sage Extracts and Their Components

Thus, in one aspect the invention relates to a sage extract containing such tricyclic diterpene (derivative)s I, II and III, preferably to a sage extract containing such a mixture of the tricyclic diterpene (derivative)s I, II, III and IV, more preferably to a sage extract containing such a mixture of the tricyclic diterpene (derivative)s I to V, for use as medicaments for the treatment of a disorder connected to reduced neurotransmission.

In another aspect, the invention relates to the use of a sage extract containing a mixture of tricyclic diterpene (derivative)s, with the definitions and preferences as described above, for the manufacture of a composition for the treatment of a disorder connected to reduced neurotransmission, particularly for the manufacture of an antidepressant, a mood/vitality improver, a stress reliever, a condition improver, a reducer of anxiety, a reducer of obsessive-compulsive behaviour, a relaxant, a sleep improver and/or an insomnia alleviator.

In still another aspect, the invention relates to a dietary composition comprising a sage extract containing a mixture of tricyclic diterpene (derivative)s, as well as to a pharmaceutical composition comprising a sage extract containing a mixture of tricyclic diterpene (derivative)s, and a conventional pharmaceutical carrier.

Furthermore, the invention relates to a method for the treatment of a disorder connected to reduced neurotransmission in animals including humans, said method comprising administering an effective dose of a sage extract containing a mixture of tricyclic diterpene (derivative)s, to animals including humans which are in need thereof.

Animals in the context of the present invention include humans and encompass mammals, fish and birds. Preferred “animals” are humans, pet and companion animals and farm animals. Examples of pet and companion animals are dogs, cats, birds, aquarium fish, guinea pigs, (jack) rabbits, hares and ferrets. Examples of farm animals are aquaculture fish, pigs, horses, ruminants (cattle, sheep and goats) and poultry.

In the context of this invention “treatment” also encompasses co-treatment as well as prevention. “Prevention” can refer to either the first occurrence (primary prevention) or to a recurrence (secondary prevention).

The term “reduced neurotransmission” is used in the present application in accordance with its meaning well-known to the person skilled in the art and relates to a dysregulation of neurotransmission and which may occur at the level of neurotransmitter biosynthesis, processing, storage, release, re-uptake and receptor binding. Reduced neurotransmission may manifest itself in animals including humans as a disturbance of behaviour, emotions, mood and thinking processes, for example, in one of various types of depression.

Thus, the present invention is also directed to a method for the prevention of a disorder connected to reduced neurotransmission in animals including humans, said method comprising administering an effective amount of a sage extract containing a mixture of tricyclic diterpene (derivative)s with the definitions and preferences as described above, to animals including humans which are in need thereof. In this regard an effective amount of a sage extract may especially be used for maintaining mental well-being, for maintaining a balanced cognitive function, for helping to reduce the risk of mood swings, for helping to retain a positive mood and for supporting cognitive wellness, and for helping to maintain a good sleep quality.

In the context of this invention the term “disorder” also encompasses diseases.

Medicaments/compositions for the treatment of disorders connected to reduced neurotransmission encompass antidepressants, mood/vitality improvers, stress relievers, condition improvers, anxiety reducers and obsessive-compulsive behaviour reducers, relaxants, sleep improvers and/or insomnia alleviators. They all improve, enhance and support neurotransmission, especially in the central nervous system, and therefore alleviate mental dysfunction.

Antidepressants are medicaments/compositions for treating mental-, behavioural- and emotional/affective-, neurotic-, neurodegenerative-, eating- and stress-related- disorders, such as unipolar depression, bipolar depression, acute depression, chronic depression, subchronic depression, dysthymia, postpartum depression, premenstrual dysphoria/syndrome (PMS), climacteric depressive symptoms, aggression, attention deficit disorders, social anxiety disorders, seasonal affective disorders and anxiety (disorders), such as GAD, fibromyalgia syndrome, post-traumatic stress disorders, panic disorders and obsessive-compulsive disorders, restless leg syndrome, nervousness, migraine/primary headaches and pain in general, emesis, bulimia, anorexia nervosa, binge eating disorder, gastrointestinal disorders, burn-out syndrome and irritability.

Antidepressants can also be used for (the manufacture of compositions for) primary and secondary prevention and/or the treatment of neurocognitive impairment. Furthermore they are also effective in the treatment of depressive symptoms or other symptoms related to disturbed neurotransmission occurring as comorbidity in chronic diseases such as cardiovascular diseases, strokes, cancer, Alzheimer's disease, Parkinson's disease, and others.

The sage extracts containing mixtures of tricyclic diterpene derivatives and dietary/pharmaceutical compositions containing them are thus suitable for the treatment of animals including humans.

In a further embodiment of the present invention sage extracts containing mixtures of tricyclic diterpene (derivative)s with the definitions and preferences as described above find use as mood improvers in general as well as for the manufacture of compositions (such as dietary/pharmaceutical compositions, food, beverages) for such use. “Mood improver”, “emotional wellness booster” or “vitality improver” means that the mood of a person treated with it is enhanced, that the self-esteem is increased and/or that negative thoughts and/or negative tension are/is reduced. It also means that the emotions are balanced and/or that general, especially mental, well-being and vitality is improved or maintained, as well as that the risk of mood swings is (helped to be) reduced and that a positive mood is (helped to be) retained.

The sage extracts containing mixtures of tricyclic diterpene (derivative)s with the definitions and preferences as described above can also be used in general as anxiety reducers and/or obsessive-compulsive behaviour reducers for animals including humans; preferably for humans, pet animals and farm animals.

“Anxiety reducer” means that chronic tension and anxious worrying and tension are lessened or alleviated. Hypervigilance syndrome, including restlessness, muscle tension and sleep problems, are reduced or relieved. Social- and other phobias are resolved. In general, the social environment is experienced as less threatening. The person is emotionally relaxed, experiences comfort and enjoys company and contact with other people.

“Relaxant”, “sleep improver” or “insomnia alleviator” means improving sleep onset and helping a person to easily enter sleep, to maintain undisrupted sleep throughout the night. It also means that circadian rhythm-associated sleep disturbances, due to jet-lag or shift work, are corrected and symptoms associated with sleeplessness, i.e. impairment of cognitive function and memory, mental and physical fatigue, dreaminess, are abolished or relieved and the overall quality of life and vital energy are improved.

Moreover, sage extracts containing mixtures of tricyclic diterpene (derivative)s with the definitions and preferences as described above, as well as compositions comprising an effective dose of them, are useful for the treatment, prevention and alleviation of stress-related symptoms, for the treatment, prevention and alleviation of symptoms related to work overload, exhaustion and/or burn-out, for the increase of resistance or tolerance to stress and/or to favour and facilitate relaxation in normal healthy individuals i.e. such compositions have an effect as “stress relievers”.

Furthermore, sage extracts containing mixtures of tricyclic diterpene (derivative)s with the definitions and preferences as described above, as well as compositions comprising an effective dose of them, are useful for the treatment, prevention and alleviation of anxiety and obsessive-compulsive behaviour in humans and animals.

A further embodiment of the present invention relates to the use of sage extracts containing mixtures of tricyclic diterpene (derivative)s with the definitions and preferences as described above, and to the use of compositions containing them (such as dietary/pharmaceutical compositions, food, beverages) as “condition improvers”, i.e. as means to reduce irritability and tiredness, to reduce, prevent or alleviate physical and mental fatigue, and to increase energy in more general terms, especially to increase brain energy production, in diseased or normal healthy individuals. Moreover for cognition improvement in general, and especially for maintenance or improvement of attention and concentration, of memory and the capacity for remembering, of learning ability, language processing, problem solving and intellectual functioning; for improvement of short-term memory; for increasing mental alertness; for enhancing mental vigilance; for reducing mental fatigue; for supporting cognitive wellness, for maintaining balanced cognitive function, for the regulation of hunger and satiety and for the regulation of motor activity.

### Veterinary Uses

Pets and farm animals can be in conditions in need of enhanced or improved neurotransmission, which can be provided by the present invention. Animals may exhibit adverse behavioural and/or physiological reactions to stressful situations; animals raised in mass production environments, or being transported under unfavourable conditions, can display a decline in meat or milk quantity or quality; stressed poultry can resort to feather-picking, reduced egg laying and cannibalism. Many animals can become aggressive or display stereotypic-, anxiety- and obsessive-compulsive-behaviours under adverse housing or transport conditions.

Thus, another aspect of this invention is veterinary uses of sage extracts containing mixtures of tricyclic diterpene (derivative)s, with the definitions and preferences as described above, as dietary/pharmaceutical compositions.

In a preferred embodiment of the present invention, sage extracts containing mixtures of tricyclic diterpene (derivative)s, with the definitions and preferences as described above, are administered for preventing stress in farm animals and mass production livestock husbandry, during transport to slaughter and/or for preventing loss of quality of meat of said farm animals under such circumstances. The farm animals are preferably poultry, cattle, sheep, goats and swine.

In another preferred embodiment of the present invention, sage extracts containing mixtures of tricyclic diterpene (derivative)s, with the definitions and preferences as described above, are administered to poultry for preventing feather-picking and cannibalism resulting in, for example, loss of meat quality and egg production.

Another aspect of this invention is a method for preventing and/or alleviating stress in aquaculture, comprising administering sage extracts containing mixtures of tricyclic diterpene (derivative)s, with the definitions and preferences as described above, to animals which are in need thereof, wherein the animals are fish or shrimps.

In another preferred embodiment of the present invention, sage extracts containing mixtures of tricyclic diterpene (derivative)s, with the definitions and preferences as described above, are administered to pets or companion animals for reduction of stress, tension and aggressiveness and compulsive behaviour exhibited under stressful conditions, such as separation, change or loss of owner, during holiday separation and husbandry in so-called “animal hotels” and husbandry in animal shelters or refuges.

Still another aspect of this invention is a method for preventing/reducing symptoms associated with stressful conditions in animals used in the fur industry, preferably minks, foxes and hares.

### Preferred Doses and Compositions of the Present Invention

For humans a suitable daily dosage of a sage extract with the definitions and preferences as described above, for the purposes of the present invention, may be so that the mixture of tricyclic diterpene (derivative)s is within the range of from 0.001 mg per kg body weight to about 20 mg per kg body weight per day. More preferred is a daily dosage of a mixture of tricyclic diterpene (derivative)s from about 0.01 to about 10 mg per kg body weight, and especially preferred is a daily dosage of a mixture of tricyclic diterpene (derivative)s from about 0.05 to 5.0 mg per kg body weight.

The term “dietary compositions” comprises any type of (fortified) food/feed and beverages, also including clinical nutrition and dietary supplements. The dietary compositions according to the present invention may further contain protective hydrocolloids, binders, film-forming agents, encapsulating agents/materials, wall/shell materials, matrix compounds, coatings, emulsifiers, surface active agents, solubilising agents (oils, fats, waxes, lecithins etc.), adsorbents, carriers, fillers, co-compounds, dispersing agents, wetting agents, processing aids (solvents), flowing agents, taste-masking agents, weighting agents, jellyfying agents, gel-forming agents, antioxidants and antimicrobials.

Beside a pharmaceutically acceptable carrier and a sage extract containing a mixture of tricyclic diterpene (derivative)s with the definitions and preferences as described above, the pharmaceutical compositions according to the present invention may further contain conventional pharmaceutical additives and adjuvants, excipients or diluents, flavouring agents, preservatives, stabilisers, emulsifying agents, buffers, lubricants, colorants, wetting agents, fillers, and the like. The carrier material can be organic or inorganic inert carrier material suitable for oral/parenteral/injectable administration.

The dietary and pharmaceutical compositions according to the present invention may be in any galenic form that is suitable for administering to the animal body including the human body, especially in any form that is conventional for oral administration, e.g. in solid form such as (additives/supplements for) food or feed, food or feed premix, fortified food or feed, tablets, pills, granules, dragées, capsules, and effervescent formulations, such as powders and tablets, or in liquid forms such as solutions, emulsions or suspensions as e.g. beverages, pastes and oily suspensions. The pastes may be filled into hard or soft shell capsules. Examples for other application forms are those for transdermal, parenteral or injectable administration, The dietary and pharmaceutical compositions may be in the form of controlled (delayed) release formulations.

Examples for fortified food are cereal bars and bakery items such as cakes and cookies.

Beverages encompass non-alcoholic and alcoholic drinks as well as liquid preparations to be added to drinking water and liquid food, Non-alcoholic drinks are e.g. soft drinks, sport drinks, fruit juices, lemonades, near-water drinks (i.e. water-based drinks with a low calorie content), teas and milk-based drinks. Liquid foods are e.g. soups and dairy products (e.g. muesli drinks).

In solid dosage unit preparations for humans, a mixture of tricyclic diterpene (derivative)s is suitably present in an amount in the range of from about 0.1 mg to about 1000 mg, preferably in the range of from about 1 mg to about 500 mg, per dosage unit. The amount of a sage extract containing such a mixture of tricyclic diterpene (derivative)s with the definitions and preferences as described above can be calculated accordingly.

In dietary compositions, especially in food and beverages for humans, a mixture of tricyclic diterpene (derivative)s is suitably present in an amount in the range of from about 0.0001 (1 mg/kg) to about 5 weight-% (50 g/kg), preferably from about 0.001 (10 mg/kg) to about 1 weight-%, (10 g/kg) more preferably from about 0.01 (100 mg/kg) to about 0.5 weight-% (5 g/kg), based upon the total weight of the food or beverage. The amount of a sage extract containing such a mixture of tricyclic diterpene (derivative)s can be calculated accordingly.

In food and drinks in a preferred embodiment of the invention, the amount of a mixture of tricyclic diterpene (derivative)s is in the range of from 10 to 30 mg per serving, i.e. 120 mg per kg food or drink. The amount of a sage extract containing such a mixture of tricyclic diterpene (derivative)s can be calculated accordingly.

For animals excluding humans a suitable daily dosage of a mixture of tricyclic diterpene (derivative)s, for the purposes of the present invention, may be within the range of from 0.001 mg per kg body weight to about 1000 mg per kg body weight per day. More preferred is a daily dosage in the range of from about 0.1 mg to about 500 mg per kg body weight, and especially preferred is a daily dosage in the range of from about 1 mg to 100 mg per kg body weight. The amount of a sage extract containing such a mixture of tricyclic diterpene (derivative)s can be calculated accordingly.

The invention is illustrated further by the following examples.

### EXAMPLES

Three different sage extracts were used in the experiments described below.

Sage Extract A was obtained from FLAVEX Naturextrakte GmbH, Rehlingen, Germany (“Salbei Extrakt, Typ Nr. 063.001”). It contained 0.06 weight-% of carnosol, 0.14 weight-% of carnosic acid and 0.36 weight-% of carnosic acid 12-methyl ether, based on the total weight of the sage extract and measured by HPLC-UV (High Pressure Liquid Chromatography-Ultraviolet) at 210 nm with the pure substances as reference.

Sage Extract B was obtained from FLAVEX Naturextrakte GmbH, Rehlingen, Germany (“Salbei Antioxidans Extrakt, Typ Nr. 063.007”). It contained 3.50 weight-% of carnosol, 10.70 weight-% of carnosic acid, 4.30 weight-% of carnosic acid 12-methyl ether, 0.3 weight-% of 20-deoxocarnosol and 0.3 weight-% of rosmanol, based on the total weight of the sage extract and measured by HPLC-UV at 210 nm with the pure substances as reference. The extract was produced as follows: Dried Salvia officinalis leaves (raw material) were mechanically reduced to small pieces, which were then placed a pressure-stable extraction vessel. Then super-critical CO₂ was passed through the extraction vessel at a temperature of 50° C., a high pressure of 280 bar and in the presence of a small amount of ethanol, thereby dissolving the lipophilic compounds. In the separator the dissolved compounds (the extract) were separated by a pressure reduction to 60 bar at a temperature of 30° C. The CO₂ was recycled back into the process. Approximately 1 kg of sage extract B can be obtained from 14 to 20 kg of dried Salvia officinalis leaves.

Sage Extract C contained 7.44 weight-% of carnosol, 1.56 weight-% of carnosic acid, 0.23 weight-% of carnosic acid 12-methyl ether, below 0.1 weight-% of 20-deoxocarnosol and 0.41 weight-% of rosmanol, based on the total weight of the sage extract and measured by HPLC-UV at 210 nm with the pure substances as reference. Sage extract C was manufactured according to the following procedure: 700 g of the dried biomaterial (Salvia officinalis leaves) was extracted with methanol and then evaporated to dryness. Subsequent solvent-solvent partitionings by use of hexane: H₂O followed by methyl tert-butyl ether (MTBE):H₂O were performed. The MTBE partition was evaporated to dryness, 40 g of this extract was obtained which yielded enriched amounts of abietanes and triterpenes.

The actions of the monoamine neurotransmitters, serotonin, dopamine and noradrenaline, are regulated through their rapid uptake and clearance from synaptic junctions by plasma membrane transport proteins. The monoamine transporters in central monoaminergic neurones are responsible for the recovery of up to 90% of released neurotransmitter and are high affinity targets for a number of psychoactive agents such as cocaine, amphetamine and antidepressants. These agents, by blocking transporters and consequently preventing neuronal uptake, elevate levels of extracellular neurotransmitter concentrations in both the central and peripheral nervous systems, contributing to their behavioural and autonomic effects. Thus, inhibition of uptake of serotonin, dopamine and noradrenaline by one or more of the sage extracts is illustrated by the following three examples.

### Example 1

### Serotonin Uptake Inhibition by Sage Extracts Containing Tricyclic Diterpene (Derivative)s

Human embryonic kidney (HEK-293) cells stably expressing the human serotonin re-uptake transporter (hSERT) were obtained from R. Blakely, Vanderbilt University, USA. The cells were routinely grown in Dulbecco's Modified Eagle's Medium (Bioconcept) containing 10% dialysed foetal calf serum (Invitrogen), penicillin, streptomycin, L-glutamine and the antibiotic G418 and passaged by trypsinisation. On the day of assay, cells from 80% confluent flasks were harvested by gentle washing with warm phosphate buffered saline (PBS). Cells were then washed once by centrifugation and re-suspended in Krebs-Ringer bicarbonate buffer (Sigma) supplemented with 35 μM pargyline, 2.2 mM CaCl₂, 1 mM ascorbic acid and 5 mM N-2-hydroxyethylpiperazine-N′-2-ethanesulfonic acid (HEPES buffer) at a concentration of 10,000 cells in 160 μl of buffer, and aliquoted into round-bottomed polypropylene 96-well microtitre plates (Corning) at 10,000 cells per well. Serotonin uptake into the cells was determined by addition of radio-labelled [3H] serotonin (GE Healthcare) at a concentration of 20 nM, and incubation for 40 minutes at 37° C. with gentle shaking. At the end of this time unincorporated label was removed by filtration though Unifilter 96 GF/B plates (Perkin Elmer) using a Tomtec Mach III M cell harvester. The incorporated serotonin retained on the plates was quantified by liquid scintillation counting using Microscint-40/Topcount (Perkin Elmer).

The effect of sage extracts A, B and C on serotonin uptake was determined by their inclusion in the assay at a range of concentrations between 0.00316 and 100 μg/ml for 10 minutes prior to and during the addition of [³H] serotonin. The SSRI, fluoxetine, (0.03 nM −1 μM) was used as reference compound. Serotonin uptake via the transporter was inhibited by sage extracts A, B and C in a concentration-dependent manner. The calculated IC₅₀ values for inhibition of serotonin uptake by sage extracts A, B and C are shown in Table 1.

TABLE 1
Inhibition of serotonin uptake into transfected HEK-293 cells by sage
extracts A, B and C and fluoxetine.

| Substance | IC₅₀ for Tritiated Serotonin Uptake | |
|---|---|---|
| Sage Extract A | 19.0 ± 2.8 | μg/ml (n = 2) |
| Sage Extract B | 11.3 ± 2.3 | μg/ml (n = 3) |
| Sage Extract C | 23.5 ± 7.3 | μg/ml (n = 2) |
| Fluoxetine | 5 | nM |

Data is shown as mean ± s.e.m., where multiple assays were performed.

### Example 2

### Dopamine Uptake Inhibition by Sage Extract B

Chinese hamster ovary (CHO)-K1 cells expressing the human dopamine transporter (hDAT) were plated before the assay. Cells (2×10⁵/ml) were incubated with sage extract B and/or vehicle in modified Tris-HEPES buffer (5 mM Tris-HCl, 7.5 mM HEPES, pH 7.1), further containing 120 mM NaCl, 5.4 mM KCl, 1.2 mM CaCl₂, 1.2 mM MgSO₄, 5 mM D-glucose and 1 mM ascorbic acid, at 25° C. for 20 minutes before addition of 50 nM [³H]-dopamine for 10 minutes. Specific signal was determined in the presence of 10 μM nomifensine (dopamine reuptake inhibitor). Cells were then solubilised with 1% SDS lysis buffer. Reduction of [³H]-dopamine uptake by 50 per cent or more 50%) relative to vehicle controls indicated significant inhibitory activity. Sage extract B was screened at 10 concentrations (0.00316-100 μg/ml), these same concentrations being concurrently applied to a separate group of cells and evaluated for possible compound-induced cytotoxicity only if significant inhibition of uptake was observed. Nomifensine (0.001-0.1 μM) was used as reference compound.

TABLE 2
Measured IC₅₀ values for inhibition of dopamine reuptake into
transfected CHO-K1 cells by sage extract B and the reference compound,
nomifensine.

| Compound | IC₅₀ |
|---|---|
| Sage extract B | 18.6 μg/ml |
| Nomifensine | 0.0104 μM |

### Example 3

### Noradrenaline Uptake Inhibition by Sage Extract B

Madin Darby canine kidney (MDCK) cells, stably expressing the human noradrenaline transporter (hNAT) were plated one day before the assay. The cells (2×10⁵/ml) were preincubated with sage extract B and/or vehicle in modified Tris-HEPES buffer (5 mM Tris-HCl, 7.5 mM HEPES, pH 7.1), further containing 120 mM NaCl, 5.4 mM KCl, 1.2 mM CaCl₂, 1.2 mM MgSO₄, 5 mM D-glucose and 1 mM ascorbic acid, at 25° C. for 20 minutes, then 25 nM [³H]-noradrenaline was added for 10 minutes incubation. Cells in each well were then rinsed twice, solubilised with 1% SDS lysis buffer and the lysate was analysed to determine [³H]-noradrenaline uptake. Specific signal was determined in the presence of 10 μM desipramine (tricyclic antidepressant which inhibits noradrenaline reuptake). Reduction of [³H]-noradrenaline uptake by 50 percent or more (≧50%) relative to vehicle controls indicated significant inhibitory activity. Sage extract B was screened at ten concentrations (0.00316-100 μg/ml), these same concentrations being concurrently applied to a separate group of cells and evaluated for possible compound-induced cytotoxicity only if significant inhibition of uptake was observed. Desipramine (0.5-50 nM) was used as reference compound.

TABLE 3
Measured IC₅₀ values for inhibition of noradrenaline reuptake into
transfected MDCK cells by sage extract B and the reference compound,
desipramine.

| Compound | IC₅₀ |
|---|---|
| Sage extract B | 32.2 μg/ml |
| Desipramine | 1.93 nM |

Therefore, sage extract B was demonstrated to have triple-reuptake inhibitory effects, through its inhibition of serotonin, dopamine and noradrenaline in the in vitro assays as described above.

### Example 4

### Monoamine Oxidase Inhibition by Sage Extracts Containing Tricyclic Diterpene (Derivative)s

The organic amines p-tyramine or benzylamine were used as substrates for the monoamine oxidase A (MAO-A) and B (MAO-B) enzymes respectively. The H₂O₂ produced by this reaction was quantified by reaction with vanillic acid, catalysed by horse radish peroxidase (HRP).

The reactions were carried out at 37° C. in polystyrene microtitre plates. The MAO enzymes (final concentration 2 U/ml) were mixed with either p-tyramine (Sigma, final concentration 0.5 mM) or benzylamine (Sigma, final concentration 0.5 mM) as appropriate and the chromogenic solution (containing vanillic acid (Fluka), 4-aminoantipyrine (Fluka) and horse radish peroxidase (Sigma), final concentrations 0.25 mM, 0.125 mM and 1 U/ml respectively) in 0.2 M potassium phosphate buffer, pH 7.6. After 40 min incubation, plates were analysed in a microtitre plate absorbance reader e.g. Spectramax M5 (Molecular Devices Corporation), at 495 nm.

The effect of sage extracts A, B and C on the monoamine oxidase enzymes was determined by their inclusion in the assay at a range of concentrations between 0.03 and 100 μg/ml for 10 minutes prior to and during the incubation with substrate. To determine the effect of the extracts on the HRP-catalysed portion of the reaction, the MAO enzyme was replaced by H₂O₂ (Molecular Probes, final concentration 50 μM), The reactions containing MAO-A and MAO-B were both inhibited by sage extracts B and C in a concentration-dependent manner, whilst the control reaction was unaffected. The measured IC₅₀ values for inhibition of monoamine oxidase activity by sage extracts A, B and C are shown in Table 4.

TABLE 4
Inhibition of MAO-A and MAO-B by sage extracts A, B and C.

| | IC₅₀ for | | IC₅₀ for | |
|---|---|---|---|---|
| Substance | Inhibition of MAO-A | | Inhibition of MAO-B | |
| Sage Extract | >>100 | μg/ml (n = 2) | >>100 | μg/ml (n = 2) |
| A | | | | |
| Sage Extract | 3.66 ± 0.36 | μg/ml (n = 2) | 7.9 ± 0.9 | μg/ml (n = 2) |
| B | | | | |
| Sage Extract | 5.7 ± 0.3 | μg/ml (n = 3) | 18.9 ± 1.5 | μg/ml (n = 2) |
| C | | | | |

Results are shown as mean ± s.e.m.

### Example 5

### Effects of Sage Extract B on Behaviour in the Marble Burying Test in the Mouse

“Defensive burying” behaviour was demonstrated by rats burying noxious objects, such as drinking spouts filled with an unpleasant-tasting liquid (Wilkie, et al 1979 J. Exp. Anal. Behav. 31:299-306.) or shock prods (Pinel, et al 1978 J. Comp. Phys. Psych. 92:708-712.). The marble burying test was devised as a modification of such a test (Poling et al. 1981 J Exp. Anal. Behav. 35:31-44.). Rats were exposed to individual cages, each containing 25 marbles, daily for 10 or 21 consecutive days. The number of marbles buried, on each day of the 10 d period, or 24 h after the 21 d exposure, were counted. The authors reported that the burying of marbles was not determined by novelty, or due to any noxious stimuli.

Marble burying behaviour by mice is reported to be sensitive to a range of minor (e.g. diazepam) and major (e.g. haloperidol) tranquilisers (Broekkamp et 1986 Eur. J. Pharm. 126:223-229), in addition to SSRIs (e.g. fluvoxamine, fluoxetine, citalopram), tricyclic antidepressants (e.g. imipramine, desipramine) and selective noradrenaline reuptake inhibitors (e.g. reboxetine), at doses which do not induce sedation. The model may reflect either anxiety-like- or obsessive-compulsive-behaviour (see De Boer et al 2003 Eur, J. Pharm. 463:145-161.).

The method applied here follows that described by Broekkamp et al. (1986). Mice (n=15 per treatment group) were individually placed in transparent plastic cages (33×21×18 cm) with 5 cm of sawdust on the floor and 25 marbles (diameter 1 cm) grouped in the centre of the cage. A second, up-turned, cage served as a lid. The number of marbles covered by sawdust (by at least two-thirds) was counted at the end of the 30-min test period. Tests were performed by investigators blind to the drug treatment protocol.

Prior to testing, all test cages and marbles were “impregnated” by leaving 10 naive mice in each cage for 15 minutes.

Sage extract B (30, 100, 300 mg/kg, p.o., 24, 5 and 1 h prior to the test) was thus investigated in the marble burying test to evaluate its potential anxiolytic effects in the mouse. Clobazam, fluoxetine and venlafaxine were used as reference compounds.

TABLE 5
Effects of sage extract B, venlafaxine, fluoxetine and clobazam in the
marble burying test in the mouse (15 mice per group).

| | | NUMBER OF MARBLES | | |
|---|---|---|---|---|
| | | COVERED BY SAWDUST | | |
| TREATMENT | | | | % |
| (mg/kg) | TREATMENT | | | change |
| p.o. −24 h | (mg/kg) | | p | from |
| and −5 h | p.o. −1 h | mean ± s.e.m. | value | control |
| Vehicle | Vehicle | 17.9 ± 2.1 | — | — |
| sage extract | sage extract B (30) | 14.0 ± 2.3NS | 0.2266 | −22% |
| B (30) | | | | |
| sage extract | sage extract B (100) | 14.9 ± 2.2NS | 0.3419 | −17% |
| B (100) | | | | |
| sage extract | sage extract B (300) | 10.6 ± 2.6* | 0.0374 | −41% |
| B (300) | | | | |
| Venlafaxine | Venlafaxine (24)# | 9.5 ± 1.8** | 0.0058 | −47% |
| (24) | | | | |
| Fluoxetine (10) | Fluoxetine (10) | 7.6 ± 1.6*** | 0.0006 | −58% |
| Vehicle | Clobazam (32) | 2.9 ± 1.2*** | <0.0001 | −84% |

#dead after the first administration ( 1/15) and after the second administration ( 1/15).
Data is shown as mean ± s.e.m. and as % change from control, where NS = non-significant, *p < 0.05, **p < 0.01, ***p < 0.001 (Student's t-test).

The highest dose of sage extract B tested (300 mg/kg) clearly and significantly reduced marble burying behaviour, in a similar manner to the serotonin and noradrenaline re-uptake inhibitor venlafaxine.

### Example 6

### Effects of Sage Extract B on Behaviour in the Light-Dark Box Test in the Mouse

The method, which detects anxiolytic activity, follows that described by Crawley (1981, Pharmacol. Biochem. Behav., 15:695-699). Anxiolytics increase the time spent in the light compartment.

Animals were placed in the light compartment of a two-compartment box with one half light and open (25×27×27 cm) and the other half dark and closed (20×27×27 cm). The time spent in each compartment as well as the number of times the animal crossed from one side to the other was scored during a 3-min test. 15 mice were studied per group. The test was performed blind.

Sage extract B, dispersed in corn oil, was evaluated at 3 doses (30, 100, 300 mg/kg, p.o., 24, 5 and 1 h prior to the test) and compared with a vehicle control group (corn oil). Clobazam (32 mg/kg p.o., dispersed in 0.2% w/v hydroxypropylmethyl cellulose in distilled water), administered 1 h before the test, was used as reference substance. Mice in this group received additional administrations of vehicle at 24 h and 5 h before the test in order to maintain experimental blinding.

TABLE 6
Effects of sage extract B (SAB) and clobazam in the light-dark box test in the mouse (15 mice per group).

| | | | | | TIME SPENT IN LIGHT | | |
|---|---|---|---|---|---|---|---|
| | | NUMBER OF CROSSINGS | | | COMPARTMENT (s) | | |
| TREATMENT | TREATMENT | | | % change | | | % change |
| (mg/kg) | (mg/kg) | | p | from | | p | from |
| p.o. −24 h, −5 h | p.o. −1 h | mean ± s.e.m. | value | control | mean ± s.e.m. | value | control |
| Vehicle | Vehicle | 11.2 ± 1.3 | — | — | 71.7 ± 4.9 | — | — |
| SAB (30) | SAB (30) | 12.8 ± 1.6 NS | 0.4407 | +14% | 73.7 ± 5.3 NS | 0.7909 | +3% |
| SAB (100) | SAB (100) | 9.7 ± 1.0 NS | 0.3739 | −13% | 70.3 ± 6.5 NS | 0.8582 | −2% |
| SAB (300) | SAB (300) ^{#1 #2} | 12.6 ± 1.7 NS | 0.5133 | +13% | 101.1 ± 11.0 * | 0.0187 | +41% |
| Vehicle | Clobazam (32) ^{#2} | 11.9 ± 1.8 NS | 0.7405 | +6% | 125.5 ± 6.4 *** | <0.0001 | +75% |

Data is shown as mean ± s.e.m. and % change from control, where
NS = non-significant,
* p < 0.05,
*** p < 0.001 (Student's t-test).
^{#1} dead after p.o. −1 h administration (false administration) (1/15).
^{#2} sedation; sage extract B at 300 mg/kg (1/14) and clobazam (2/15).

The highest dose of sage extract B tested (300 mg/kg) clearly and significantly increased the time spent in the light compartment compared to vehicle control. Thus, the sage extract B demonstrated anxiolytic-like activity at 300 mg/kg.

### Example 7

### Preparation of a Soft Gelatine Capsule

A soft gelatine capsule is prepared comprising the following ingredients:

| Ingredient | Amount per Capsule |
|---|---|
| A mixture containing carnosic acid, | 200 mg |
| carnosol and carnosic acid 12-methyl ether | |
| Lecithin | 50 mg |
| Soy bean oil | 250 mg |

Two capsules per day for 3 months may be administered to a human adult for the treatment of mild chronic dysthymia.

### Example 8

### Preparation of a Soft Gelatine Capsule

A soft gelatine capsule is prepared comprising the following ingredients:

| | Amount per |
|---|---|
| Ingredient | Capsule |
| A mixture containing carnosic acid, carnosol | 200 mg |
| and carnosic acid 12-methyl ether | |
| Evening primrose oil | 300 mg |
| Vitamin B₆ | 100 mg |

One capsule per day, preferably during the second half of the menstrual cycle, should be taken for 14 days for the treatment of premenstrual syndrome and premenstrual dysphoric disorder.

### Example 9

### Preparation of a Tablet

A tablet is prepared comprising the following ingredients:

| Ingredient | Amount per tablet |
|---|---|
| A mixture containing carnosic acid, | 100 mg |
| carnosol and carnosic acid 12- | |
| methyl ether | |
| Passion flower standardised extract | 150 mg |
| Green Tea Extract, e.g. TEAVIGO ® | 150 mg |
| from DSM Nutritional Products, | |
| Kaiseraugst, Switzerland | |

For general well-being, energising and stress alleviation, one tablet is taken twice daily for 3 months.

### Example 10

### Preparation of an Instant Flavoured Soft Drink

| Ingredient | Amount [g] |
|---|---|
| A mixture containing carnosic acid, carnosol and | 0.9 |
| carnosic acid 12-methyl ether | |
| Sucrose, fine powder | 922.7 |
| Ascorbic acid, fine powder | 2.0 |
| Citric acid anhydrous powder | 55.0 |
| Lemon flavour | 8.0 |
| Trisodium citrate anhydrous powder | 6.0 |
| Tricalciumphosphate | 5.0 |
| β-Carotene 1% CWS from DNP AG, Kaiseraugst, | 0.4 |
| Switzerland | |
| Total amount | 1000 |

All ingredients are blended and sieved through a 500 μm sieve. The resulting powder is put in an appropriate container and mixed in a tubular blender for at least 20 minutes, For preparing the drink, sufficient water is added to 125 g of the obtained mixed powder to make up to one liter of beverage.

The ready-to-drink soft drink contains ea. 30 mg of the tricyclic diterpene (derivative)s mixture per serving (250 ml). As a strengthener and for general well-being 2 servings per day (240 ml) should be drunk.

### Example 11

### Preparation of a Fortified Non-Baked Cereal Bar

| Ingredient | Amount [g] |
|---|---|
| A mixture containing carnosic acid, | 0.95 |
| carnosol and carnosic acid 12-methyl ether | |
| Sugar | 114.55 |
| Water | 54.0 |
| Salt | 1.5 |
| Glucose syrup | 130.0 |
| Invert sugar syrup | 95.0 |
| Sorbitol Syrup | 35.0 |
| Palm kernel fat | 60.0 |
| Baking fat | 40.0 |
| Lecithin | 1.5 |
| Hardened palm-oil | 2.5 |
| Dried and cut apple | 63.0 |
| Cornflakes | 100.0 |
| Rice crispies | 120.0 |
| Wheat crispies | 90.0 |
| Roasted hazelnut | 40.0 |
| Skimmed milk powder | 45.0 |
| Apple flavour 74863-33 | 2.0 |
| Citric acid | 5.0 |
| Total amount | 1000 |

The tricyclic diterpene (derivative)s mixture is premixed with skimmed milk powder and placed in a planetary bowl mixer. Cornflakes and rice crispies are added and the total is mixed gently. Then the dried and cut apples are added. In a first cooking pot sugar, water and salt are mixed in the amounts given above (solution 1). In a second cooking pot glucose, invert- and sorbitol- syrup are mixed in the amounts given above (solution 2). A mixture of baking fat, palm kernel fat, lecithin and emulsifier is the fat phase. Solution 1 is heated to 110° C. Solution 2 is heated to 113° C. and then cooled in a cold water bath. Afterwards solutions 1 and 2 are combined. The fat phase is melted at 75° C. in a water bath, then added to the combined mixture of solutions 1 and 2. Apple flavour and citric acid are added to the liquid sugar-fat mix. The liquid mass is added to the dry ingredients and mixed well in the planetary bowl mixer. The mass is put on a marble plate and rolled to the desired thickness, then cooled down to room temperature and cut into pieces. The non-baked cereal bar contains ca. 25 mg of the tricyclic diterpene (derivative)s mixture per serving (30 g). For general well-being and energising 1-2 cereal bars should be eaten per day.

1-10. (canceled)

11. A method for the treatment of a disorder connected to reduced neurotransmission in animals, said method comprising administering an effective dose of a composition comprising a sage extract wherein the composition comprises carnosol, carnosic acid and carnosic acid 12-methyl ether to animals which are in need thereof.

12. The method according to claim 11, wherein the animal is selected from the group consisting of a human, a pet animal or a farm animal.

13. A method according to claim 11 wherein the composition is a dietary or nutraceutical composition.

14. A method according to claim 13 wherein the composition is selected from the group consisting of: food, feed, dairy products, yoghurts, fortified food, cereal bars, bakery items, cakes, cookies, dietary supplements, tablets, pills, granules, dragées, capsules, effervescent formulations, non-alcoholic drinks, soft drinks, sport drinks, fruit juices, lemonades, near-water drinks, teas, milk-based drinks, liquid foods, soups, liquid dairy products and muesli drinks.

15. A method according to claim 11 wherein the composition is selected from the group consisting of: an antidepressant, a mood/vitality improver, a stress reliever, a condition improver, a reducer of anxiety, a reducer of obsessive-compulsive behaviour, a relaxant, a sleep improver or an insomnia alleviator.

16. A method for the treatment of a disorder connected to reduced neurotransmission in animals, said method comprising: (a) identifying a subject in need of an antidepressant, a mood/vitality improver, a stress reliever, a condition improver, a reducer of anxiety, a reducer of obsessive-compulsive behaviour, a relaxant, a sleep improver or an insomnia alleviator, and (b) administering to a subject identified in step (a) an effective amount of a composition comprising a sage extract wherein the composition comprises carnosol, carnosic acid and carnosic acid 12-methyl ether.

17. A method for the treatment of a disorder connected to increased uptake of serotonin, dopamine or noradrenaline by a monoamine neurotransmitter in animals, said method comprising administering an effective dose of a composition comprising a sage extract wherein the composition comprises carnosol, carnosic acid and carnosic acid 12-methyl ether to animals which are in need thereof, and observing an improvement.

18. A method according to claim 17, wherein the sage extract is an antidepressant, mood/vitality improver, stress reliever, condition improver, reducer of anxiety, reducer of obsessive-compulsive behavior, relaxant, sleep improver and/or insomnia alleviator.
